(19) United States
(12) Patent Application Publication
(10) Pub. No.: US 2021/0298638 A1
(43) Pub. Date: Sep. 30, 2021

(54) Method of Detection of Fatty Liver Diseases by Breath Analysis

(71) Applicant: Hygieia Sciences LLC, Johns Creek, GA (US)

(72) Inventors: Charlene Warres Bayer, Marietta, GA (US); William Hiller, Alpharetta, GA (US)

(73) Assignee: Hygieia Sciences LLC, Johns Creek, GA (US)

(21) Appl. No.: 16/832,208

(22) Filed: Mar. 27, 2020

(51) Int. Cl. A61B 5/097; A61B 5/08; A61B 5/00; A61B 5/083; G01N 30/86

(52) U.S. Cl. CPC A61B 5/097; A61B 5/082; A61B 5/4244; G01N 30/8686; A61B 5/4842; A61B 5/4845; A61B 5/0836; A61B 5/0075

(57) ABSTRACT

Analysis of a breath sample is a noninvasive point-of-care tool with ever increasing clinical applicability. Herein we describe a method to analyze the content of low-level, trace volatile organic compounds from alveolar breath captured as a breath sample from a patient suspected of having a fatty liver disease. The breath sample is then analyzed using a gas phase analysis methodology, such as gas chromatography-mass spectroscopy (“GCMS”), to generate an analysis result, such as a GCMS spectrum. A computer system is then used to develop a fingerprint pattern from the analysis result. The fingerprint pattern is then used to determine a patient status for the patient. The fingerprint pattern is typically a grouping of 75 to 450 compounds of known concentration which are indicative of a particular fatty liver disease.

### BACKGROUND

### Field of the Invention

The present invention relates generally to analysis methods for the assessment of breath samples. More specifically, the present invention relates to analysis methods for the evaluation of breath volatile organic compounds (BVOC) within a breath sample to detect fatty liver diseases within a patient.

### Description of the Related Art

Acceptance of BVOC analysis as clinical screening technique has been slow to develop. This is due to a number of issues including: unsuccessful attempts to find individual or a small number of low-level volatile organic compounds detected in breath as marker compounds for individual diseases; lack of understanding of the physiological meaning of the detected volatiles; a dearth of standardized methods and comparable results among a group of laboratories; the difficulty to adapt sophisticated technology and instrumentation for widespread acceptance in clinical settings; few larger population studies having been conducted; and inability to translate the methods and technology to clinical settings.

Some success has been achieved for tuberculosis, oxidative stress, ulcers, some lung cancer, diabetes, and tracking heart transplant rejection, but success of BVOC analysis in the clinical space has largely been limited due to the above factors. Successful screening for breast cancer has been achieved with high accuracy and precision but requires a large number of low-level BVOCs for successful screening.

### State of the Non Alcoholic Fatty Liver Disease Detection

Non-alcoholic fatty liver disease (NAFLD) is currently the most common chronic liver disease in children and adults in western countries. This is attributed to the obesity epidemic that is rising in the West. However, despite the high prevalence, and the important health consequences, diagnosing and staging NAFLD in clinical practice remains complex. Most patients remain asymptomatic until late stage disease progression, and no accurate screening tool exists in the market.

Currently available methods of detection for NAFLD include liver biopsy, serum tests, predictive scoring, and imaging. All of these methods have their limitations. Liver biopsy is extremely invasive, especially in pediatric patients, and suffers sampling errors due to the small sample taken from the most frequently used needle biopsy, typically 1/50,000^{th} of the liver. Additionally, the well-known interobserver variability of histopathology is a significant limitation to biopsy use for screening. Biopsy is not without risks; post-procedural bleeding occurs in roughly 0.5% of patients.

Serum tests suffer from their own issues. Alanine aminotransferase (ALT) is the most commonly used serum test to detect non-alcoholic fatty liver (NAFL), as ALT correlates with the presence of steatosis. However, there is broad agreement that ALT is an imperfect test to detect NAFL. Studies on the accuracy of ALT in pediatric patients is varied. Studies report sensitivity between 54% and 88% with specificity ranging between 26% and 100%. As such, diagnostic accuracy and optimal ALT cutoff for screening purposes is not established in the art. Serum tests for non-alcoholic steatohepatitis (NASH) and for fibrosis suffer similar issues as ALT.

The use of predictive scores for detection of NAFLD is divided into prediction for steatosis, NASH, and fibrosis. For steatosis, predictive scores for adult patients has reportedly good predictive value but is lacking in external validation. For pediatric patients, the predictive scores have been found to be insufficient for clinical practice. NASH predictive scores are showing good accuracy but are still lacking external validation. NASH predictive score tests for pediatric patients are currently showing mediocre AUROC for diagnosis. Predictive scores for fibrosis are accurately able to detect significant fibrosis in adults but are perform less well for detecting mild fibrosis. Tests for pediatric patients offered moderate accuracy for derivation cohorts but gave poor diagnostic accuracy in validation studies across mild and significant fibrosis. Additionally, cost and limited availability of fibrosis predictive scores is a limiting factor.

Imaging studies for NAFLD are broken down into similar categories as predictive scores; steatosis, NASH, and fibrosis. Imaging for steatosis is most commonly ultrasound with controlled attenuation parameter and magnetic resonance techniques also used. Ultrasound suffers from mediocre accuracy for detecting steatosis. False negatives, as well as false positives, are important results for clinicians to be aware of when using ultrasound for steatosis. Controlled attenuation parameter imaging is ultrasound-based technique that is showing good results, is non-invasive, and has a low cost. However, it has not been extensively validated and is not widely used at this point. Magnetic resonance techniques are very sensitive and specific in both adult and pediatric patients but suffer from limited availability and high cost. At present, magnetic resonance techniques are primarily used for research purposes in NAFLD.

Imaging for NASH is much more limited than steatosis. Presently, only multiparametric magnetic resonance imaging is used for detection of NASH. It is showing promising results for quantification of hepatic iron, inflammation, fibrosis, and fat, but only in prospective pilot studies in adult patients. Pediatric patients must wait for studies to begin, and larger prospective studies are needed for adults, in addition to the issues of cost associated with magnetic resonance imaging.

When imaging for fibrosis, elastography is the most commonly used radiological technique. Transient elastography offers good accuracy for predicting the presence of any, significant, and advanced fibrosis for both adult and pediatric patients. Patients with high BMI, as is common with NAFLD, have a high failure rate with transient elastography, which limits the use in screening for NAFLD. Ultrasound elastography allows for good sensitivity, but mediocre specificity, in pediatric patients when detecting any fibrosis. In pediatric patients with significant fibrosis, sensitivity and specificity are significantly better. Magnetic resonance elastography shows excellent accuracy in adults, but only fair accuracy in pediatric patients with any fibrosis and good accuracy for pediatric patients with significant fibrosis. In spite of this, magnetic resonance elastography is mainly a research tool because of the associated costs and availability.

Non-elastography imaging methods are used, such as contrast-enhanced ultrasound (CEUS). Pediatric studies of CEUS are currently lacking, while CEUS use in adult patients shows good results for identifying advanced fibrosis. Additionally, CEUS suffers from operator dependence, similar to histopathology limitations, and varying results between different contrast agents.

### SUMMARY

In accordance with the embodiments herein, a method for detection of fatty liver disease using breath analysis is disclosed. The method described herein generally utilizes chemical analysis of low-level BVOCs from alveolar breath captured as a breath sample from a patient. The breath sample is then analyzed using a gas phase analysis methodology to generate an analysis result, such as a gas chromatography chromatogram. A computer system is then used to develop a fingerprint pattern from the analysis result. The fingerprint pattern is then used to determine a patient status related to a specific fatty liver disease for the patient.

### BRIEF DESCRIPTION OF THE DRAWINGS

FIG. 1 provides a general overview of the method herein described.

### DETAILED DESCRIPTION OF EMBODIMENTS

In the following description, for purposes of explanation and not limitation, details and descriptions are set forth in order to provide a thorough understanding of the present invention. However, it will be apparent to those skilled in the art that the present invention may be practiced in other embodiments that depart from these details and descriptions without departing from the spirit and scope of the invention.

For the purpose of this disclosure, patient status includes diagnosis of inflammatory status, disease state, disease severity, disease progression, therapy efficacy, and changes in patient status over time. Other patient statuses are contemplated.

For the purpose of definition, fatty liver disease is one or more of non-alcoholic fatty liver, non-alcoholic steatohepatitis, and alcoholic fatty liver disease.

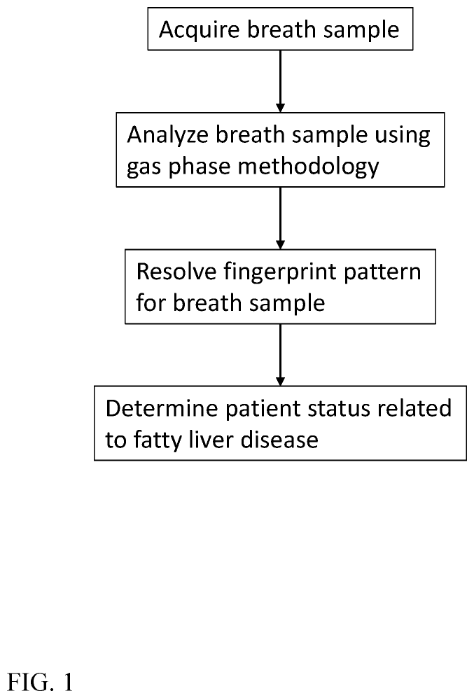

In an illustrative embodiment of the invention, as summarized in FIG. 1, the method may be summarized in the following four steps: i) collecting a breath sample from a patient in a sterile sampling vessel; ii) analyzing the breath sample using a gas phase analysis methodology to generate an analysis result; using a computer system configured to iii) resolving a fingerprint pattern from the analysis result; and iv) determining a patient status for the patient related to a fatty liver disease based on the fingerprint pattern. Typically, the fingerprint pattern will be 75 to 450 different compounds within the analysis result. It is understood, however, that smaller or larger combinations may be used as a fingerprint pattern.

In a further embodiment, the sterile sampling vessel may contain a carbon dioxide sensor. This sensor could be used to determine when the patient has reached alveolar breath. This would ensure that the breath sample contains low-level VOCs. There are a number of commercially available BVOC collection samplers available which are suitable for the present invention. Many BVOC collection samplers contain a solid-state adsorbent or thin film adsorbent media to adsorb low-level VOCs thus ensuring capture. If a solid-state adsorbent or thin film adsorbent media is used in the breath sampler, thermal desorption or thin film adsorbent media techniques are used to release the low-level VOCs from the solid-state adsorbent prior to analysis with the gas phase analysis methodology.

In a further embodiment, the gas phase analysis methodology could be gas chromatography, mass spectrometry, or gas chromatography-mass spectrometry. Other gas phase analysis methods are contemplated.

In another embodiment, the fingerprint pattern is a spectrographic or chromatographic pattern within the analysis result that is specific to a particular fatty liver disease. The fingerprint pattern is typically a group of marker compounds between 75 and 450 unique compounds in predetermined concentrations for each specific fatty liver disease. It is contemplated, however, that a single or fewer than 75 marker compound(s) could be used to identify a particular fatty liver disease.

In an additional embodiment, patient status can be inflammatory status, disease state, disease severity, disease progression, efficacy of a particular therapy, or changes in patient status over time. Other patient statuses are contemplated.

In a further embodiment, a fatty liver disease is one or more of non-alcoholic fatty liver disease and alcoholic fatty liver disease. Non-alcoholic fatty liver disease includes non-alcoholic fatty liver and non-alcoholic steatohepatitis. Other fatty liver diseases are contemplated.

The present invention address many of the issues with currently available NAFLD screening and detection by offering a noninvasive, highly accurate, inexpensive, and widely available method for the detection of steatosis, NASH, and fibrosis for both pediatric and adult patients differentiating between the three disease states. Being non-invasive and without radiological exposures is a significant improvement over the currently used modalities and will allow for frequent testing in a physician's office for both pediatric and adult subjects. Additionally, the present invention removes the interobserver error frequently found with pathology studies and some imaging modalities.

What is claimed is:

1. A method comprising:
collecting a breath sample from a patient in a sterile sampling vessel;
analyzing the breath sample using at least one gas phase analysis methodology to generate an analysis result; and
using a computer system:
resolving a fingerprint pattern from the analysis result; and
determining a patient status related to a fatty liver disease for the patient based on the fingerprint pattern.

2. The method of claim 1, wherein the sterile sampling vessel contains a carbon dioxide sensor.

3. The method of claim 1, wherein the sterile sampling vessel is a commercially available breath sampler.

4. The method of claim 1, wherein the sterile sampling vessel includes an absorbent material selected from the group consisting of solid-state adsorbent and thin film adsorbent media.

5. The method of claim 4, further comprising using thermal desorption techniques to release the breath sample from the absorbent material.

6. The method of claim 1, wherein the at least one gas phase analysis methodology is selected from the group consisting of gas chromatography, mass spectrometry, and gas chromatography-mass spectrometry.

7. The method of claim 1, wherein the fingerprint pattern is a chromatographic pattern specific to a particular fatty liver disease.

8. The method of claim 1, wherein the fingerprint pattern is a spectrographic pattern specific to a particular fatty liver disease.

9. The method of claim 1, wherein the fingerprint pattern contains at least one marker compound.

10. The method of claim 9, wherein the marker compound is a compound specific to a particular fatty liver disease.

11. The method of claim 1, wherein the patient status is selected from the group consisting of inflammatory status, disease state, disease severity, disease progression, therapy efficacy, and changes in patient status over time.

12. The method of claim 1, wherein the fatty liver disease is selected from the group consisting of non-alcoholic fatty liver disease and alcoholic fatty liver disease.

13. The method of claim 12, wherein the non-alcoholic fatty liver disease is selected from the group consisting of non-alcoholic fatty liver and non-alcoholic steatohepatitis.
